(19) United States
(12) Patent Application Publication
(10) Pub. No.: US 2012/0077257 A1
(43) Pub. Date: Mar. 29, 2012

(54) Cell measurement system

(75) Inventors: Chao-Fa Lee, Taipei (TW); Tsong-Rong Yan, Taipei (TW); Cheng-Hsing Kuo, Taipei (TW)

(73) Assignee: Tatung Company, Taipei (TW)

(21) Appl. No.: 12/929,543

(22) Filed: Feb. 1, 2011

(30) Foreign Application Priority Data
Sep. 24, 2010 (TW) 099132364

(51) Int. Cl. C12M 1/42

(52) U.S. Cl. 435/287.1

(57) ABSTRACT

A cell measurement system measures changes of frequency and transepithelial electrical resistance of a tested cell sample. The cell measurement system includes a quartz crystal sensing module, an oscillation module, a periodic wave-generation module, a low-pass filtration module, and a control module. The cell measurement system of the present invention can simultaneously measure changes of frequency and transepithelial electrical resistance of a tested cell sample during cell growth so that the growth level and healthy condition of the cells and degree of a monolayer completion of the cells can be determined.

### BACKGROUND OF THE INVENTION

1. Field of the Invention

The present invention relates to a cell measurement system and, more particularly, to a cell measurement system integrated with a quartz crystal microbalance (QCM) and a technique of measuring trans-epithelial electrical resistance (TEER), which is suitable to measure the changes of the frequency and the TEER.

2. Description of Related Art

A quartz crystal microbalance (QCM), is also known as an electronic nose, and generally used for measuring micro substances. Applying pressure on a quartz crystal will result in an induced voltage on a deformed surface of the quartz crystal. This phenomenon is known as piezoelectric effect and it is a reversible process. On the other hand, applying variable voltage to the quartz crystal will cause a physical deformation thereon.

QCMs serve to measure the mass of a substance by the piezoelectric effect and consist mainly of a quartz crystal and an oscillator circuit. The oscillator circuit is coupled to the quartz crystal to generate a resonant frequency. Because the change of loading is very small on the quartz crystal surface, the resonant frequency thereof also varies simultaneously and thus is difficult to be identified independently in liquid. Hence, conventional techniques have been trying to improve QCMs so as to detect the variation of the resonant frequency and measure the TEER.

In 1959, Sauerbrey published the relation between the mass loading on the quartz crystal surface and the resonant frequency of the quartz crystal. This relation is called the mass loading effect. Therefore, the slight change of the substance loading on the surface of the quartz crystal can be calculated by the shift of the resonant frequency of the quartz crystal. Accordingly, QCMs are widely used for accurate measurement. For example, because gas has different adsorbabilities to various adsorbents, the kind and the concentration of the gas can be determined in accordance with the mass change (which is due to adsorption of the gas) of the absorbents loading on the surface of the quartz crystal. Such methods can make QCMs to be widely used for identification odors, pollutants, toxic gases, and so on. In the aspect of detection of a fluid, QCMs can be also used to detect the viscosity of the fluid because the properties of the fluid can influence the resonant frequency of the quartz crystal.

Currently, as the techniques are improved, QCMs are gradually used as a sensor in the fields such as biological and medical sciences. However, because QCMs have their inherent limitations, the applications of QCMs are restricted thereby. For example, in the aspect of cell detection, QCMs are used to monitor the condition of the cell growth generally in accordance with the change of the resonant frequency of the quartz crystal resulted from the amount of the cell proliferation, the composition change of the culture media (owing to the consumption of the culture media by cell growth and cell secretion), and so forth. Nevertheless, the application mentioned above is to measure the amount of the cells, but not to determine whether the formed cell monolayer is integrated and has good tight junction or not. Hence, in an experiment where good integrity of the cell monolayer requires to be confirmed, QCMs can not be applied to determine the growth condition of the cell monolayer.

Accordingly, although current QCMs can measure the amount of the cell proliferation, the integrity of the cell monolayer can not be determined by QCMs. Thus, it is difficult to adopt QCMs in the related experiments where the formation of the cell monolayer needs to be determined and then the subsequent assay steps can be performed, for example, an in vitro assay for assessing drug penetration, observation of cell junction condition, a blood-brain barrier (BBB) test, drug screening test, etc. As a result, it is desired to develop a cell measurement system integrated with both the performance of measuring the amount of the cells in QCMs and the function of examining the cell monolayer, so that QCMs can be applied more widely. Moreover, researchers can monitor the conditions of the cell proliferation and the cell monolayer so as to execute subsequent assays of drug screening etc., thereby facilitating the progress of related fields.

### SUMMARY OF THE INVENTION

The object of the present invention is to provide a cell measurement system. A measuring circuit of QCMs and a technique of TEER are both integrated in the cell measurement system. Therefore, the cell measurement system can measure the changes of the frequency and the TEER owing to the changes of the cell amounts, secretion, tight junction, and so on during growth of a tested cell sample so that the growth and condition of the tested cell sample and the cell monolayer can be preliminarily detected.

To achieve the object, the present invention provides a cell measurement system, which measures changes of frequency and TEER of QCMs during the examining process. The cell measurement system includes: a quartz crystal sensing module, an oscillation module, a periodic wave-generation module, a low-pass filtration module, and a control module.

In the cell measurement system of the present invention, the quartz crystal sensing module has a first electrode, a second electrode, a quartz crystal disposed between the first and second electrodes, and a sample tank. The sample tank is used to incubate the tested cell sample and include a third electrode.

The oscillation module is coupled to the first and second electrodes of the quartz crystal sensing module to oscillate the quartz crystal thereof. Herein, the quartz crystal sensing module and the oscillation module constitute a QCM. Therefore, the second electrode of the quartz crystal sensing module can be used to detect the tested cell sample and to monitor the change of the resonance frequency of the quartz crystal, and thus the condition of the cell proliferation can be determined in the tested cell sample.

The periodic wave-generation module is coupled to the quartz crystal sensing module and has a third electrode to provide a first periodic wave. The first periodic wave is transmitted to the tested cell sample by the third electrode of the periodic wave-generation module. Herein, the second electrode of the quartz crystal sensing module is used as a receiver electrode to receive the first periodic wave output by the periodic wave-generation module. In other words, the first periodic wave is transmitted from the third electrode to the tested cell sample, and then the current passes through the cells to the second electrode and then go into the subsequent module. If the tested cell sample has good tight junction, it is relatively difficult for the current to pass through owing to the barrier of the cells and this demonstrates the higher resistance. Hence, the present invention integrates the quartz crystal sensing module and the periodic wave-generation module and makes the cell measurement system can measure the proliferation amount and the TEER of the cells at the same time. In addition, the periodic wave-generation module can be a fixed-width voltage pulse generator. The periodic wave-generation module can generate a voltage pulse of, for example, a pulse width from 20 ms to 100 ms within five seconds cyclicly. Such cycle can prevent the tested cell sample from being ionized or polarized during the operation of the cell measurement system, and the voltage pulse applied for the extremely short time will not influence the cell growth.

In the cell measurement system of the present invention, the low-pass filtration module is coupled to the periodic wave-generation module to receive the first periodic wave transmitted to the tested cell sample and outputs a second periodic wave. In the second periodic wave which passes through the low-pass filtration module and then is output, high-frequency noises are reduced in a small ratio, and thus low-frequency DC signal will outstand so that the TEER can be calculated.

The control module is coupled with the periodic wave-generation module and the low-pass filtration module to control the timing of produce the first periodic wave from the periodic wave-generation module. The control module also receives and processes the second periodic wave output from the low-pass filtration module. In accordance with this signal, the changes of the frequency and the TEER of the tested cell sample can be calculated to determine the proliferation of the tested cell sample and the integrity of the cell monolayer.

In one aspect of the present invention, the cell measurement system can further include: a power unit which supplies electricity to the oscillation module and the quartz crystal sensing module to energize the oscillation module and the quartz crystal sensing module; and a level-shift unit which is coupled to the oscillation module, the quartz crystal sensing module, and the periodic wave-generation module to shift a voltage level of the first periodic wave output by the periodic wave-generation module. Herein, in one example of the present invention, the level-shift unit is an RLC circuit in which a resistor is used to divide voltage, and a capacitor and an inductor are coupled to the oscillation module, the quartz crystal sensing module, and the periodic wave-generation module. Thus, the signals in the front and rear circuits can be coupled and the middle voltage level of the first periodic wave output from the periodic wave-generation module can be shifted down, for example, from one level range of 0-5V to another level range of −2.5-+2.5 V.

In another aspect of the present invention, the oscillation module of the cell measurement system can include: a power unit to energize the oscillation module; and the periodic wave-generation module can further include: a level-shift unit to shift a voltage level of the first periodic wave output by the periodic wave-generation module.

In further another aspect of the present invention, the oscillation module of the cell measurement system can include: a power unit to energize the oscillation module; and the cell measurement system can further include: a level-shift unit coupled to the oscillation module, the quartz crystal sensing module, and the periodic wave-generation module to shift a voltage level of the first periodic wave output by the periodic wave-generation module.

In still another aspect of the present invention, the periodic wave-generation module of the cell measurement system can include: a level-shift unit to shift a voltage level of the first periodic wave output by the periodic wave-generation module; and the cell measurement system can further include: a power unit coupled to the oscillation module and the quartz crystal sensing module to energize the oscillation module and the quartz crystal sensing module.

In addition, the cell measurement system of the present invention can further include: a frequency-monitoring module coupled to the oscillation module to monitor a frequency of a voltage level output by the oscillation module.

Furthermore, in the cell measurement system of the present invention, the control module can include an analog-to-digital converter unit to convert the analog signal to the digital signal to benefit digital demonstration of the signal.

A term “the change of the frequency” means the changes of the resonance frequency of the quartz crystal and the intensity thereof caused by cell secretion and proliferation during cell growth and it can be used for calculation of the cell amount. A term “TEER” means the resistance of the cell monolayer calculated from the voltage drop caused by the barrier of the cell monolayer when an external periodic wave is applied to the cells and it can be used to determine the integrity of the cell monolayer.

In conclusion, common QCMs generally serve to measure the micro changes of the mass. Although QCMs are gradually applied to observe cell researches including cell growth, viscosity of fluids, cell response for drug stimulation, and cell secretion, the integrity and growth condition of the cell monolayer still can not be determined by QCMs. In the cell measurement system of the present invention, one electrode in the QCM is used as a receiver electrode of measuring TEER to receive the periodic wave passing through the cells, and thus the changes of the frequency of the quartz crystal and the TEER can be monitored simultaneously. In other words, the present invention integrates two techniques of the TEER and the QCM. In addition to the original performance of the QCMs (i.e. measuring the frequency change of the quartz crystal), the performance of measuring the change of TEER is also added in the present invention, and thus the cell measurement system of the present invention can be applied to observe an assay for assessing drug penetration, cell junction condition, a blood-brain barrier (BBB) test. Therefore, the QCMs can be applied more wildly in the cell researches and provide more various bio-information.

Other objects, advantages, and novel features of the invention will become more apparent from the following detailed description when taken in conjunction with the accompanying drawings.

### BRIEF DESCRIPTION OF THE DRAWINGS

FIG. 1 shows a block diagram of the cell measurement system in Example 1 of the present invention;

FIG. 2 shows a block diagram of the cell measurement system in Example 2 of the present invention;

FIG. 3 shows a block diagram of the cell measurement system in Example 3 of the present invention;

FIG. 4 shows a block diagram of the cell measurement system in Example 4 of the present invention;

FIG. 5 shows a circuit diagram of the cell measurement system in Example 3 of the present invention; and

FIG. 6 shows an arrangement of the electrodes for measuring TEER in the cell measurement system of the examples of the present invention.

### DETAILED DESCRIPTION OF THE PREFERRED EMBODIMENT

Because of the specific embodiments illustrating the practice of the present invention, one skilled in the art can easily understand other advantages and efficiency of the present invention through the content disclosed therein. The present invention can also be practiced or applied by other variant embodiments. Many other possible modifications and variations of any detail in the present specification based on different outlooks and applications can be made without departing from the spirit of the invention.

The drawings of the embodiments in the present invention are all simplified charts or views, and only reveal elements relative to the present invention. The elements revealed in the drawings are not necessarily aspects of the practice, and quantity and shape thereof are optionally designed. Further, the design aspect of the elements can be more complex.

### Example 1

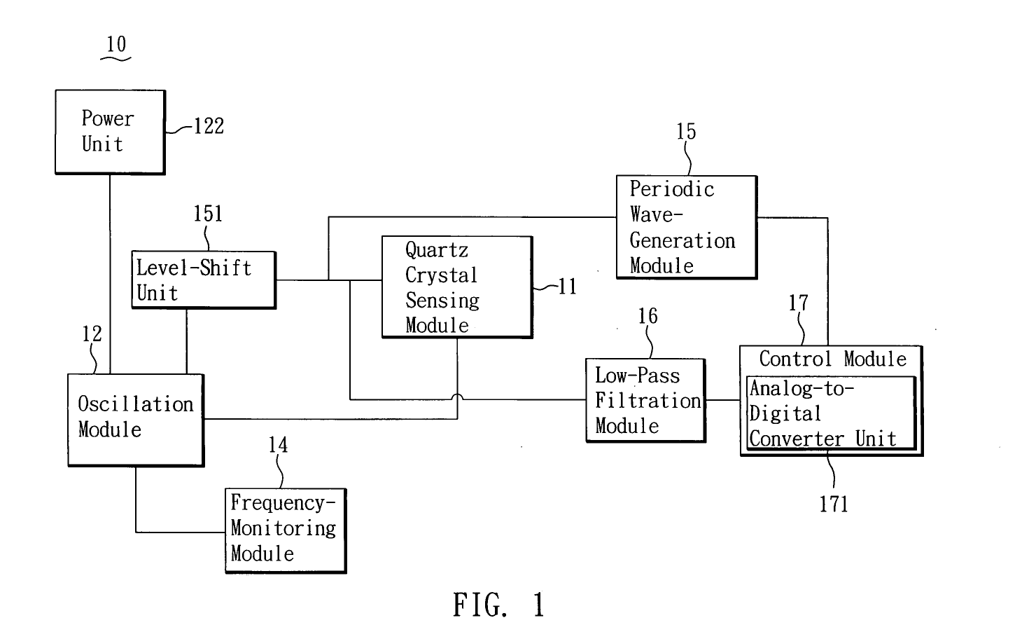

FIG. 1 shows a block diagram of a cell measurement 10 in the present example. The cell measurement 10 includes: a power unit 122, an oscillation module 12, a frequency-monitoring module 14, a level-shift unit 151, a quartz crystal sensing module 11, a periodic wave-generation module 15, a low-pass filtration module 16, and a control module 17.

The power unit 122 is coupled to the oscillation module 12. The level-shift unit 151 is coupled to the oscillation module 12, the quartz crystal sensing module 11, and the periodic wave-generation module 15. In addition, the frequency-monitoring module 14 is coupled to the oscillation module 12. When the power unit 122 energizes the oscillation module 12, the quartz crystal of the quartz crystal sensing module 11 resonates and the frequency-monitoring module 14 monitors the change of the frequency output from the oscillation module 12.

The control module 17 is coupled to the periodic wave-generation module 15 and the low-pass filtration module 16. The periodic wave-generation module 15 is coupled to the quartz crystal sensing module 11 and the low-pass filtration module 16. When the control module 17 outputs a starting signal to the periodic wave-generation module 15, the periodic wave-generation module 15 provides a first periodic wave, and the level-shift unit 151 shifts down the middle voltage level of the first periodic wave, for example, from one level range of 0-5V to another level range of −2.5-+2.5 V. Then, the first periodic wave is transmitted to a cell sample to be tested. The low-pass filtration module 16 receives the first periodic wave transmitted through the tested cell sample and outputs a second periodic wave. That is, the low-pass filtration module 16 receives a divided voltage of the first periodic wave passing through the tested cell sample. Subsequently, the control module 17 receives and processes the second periodic wave output from the low-pass filtration module 16 to calculate the changes of the frequency and the TEER owing to the tested cell sample. In the system, the control module 17 can include an analog-to-digital converter unit 171 to convert an analog signal to a digital signal for observers' convenience to record the signal.

### Example 2

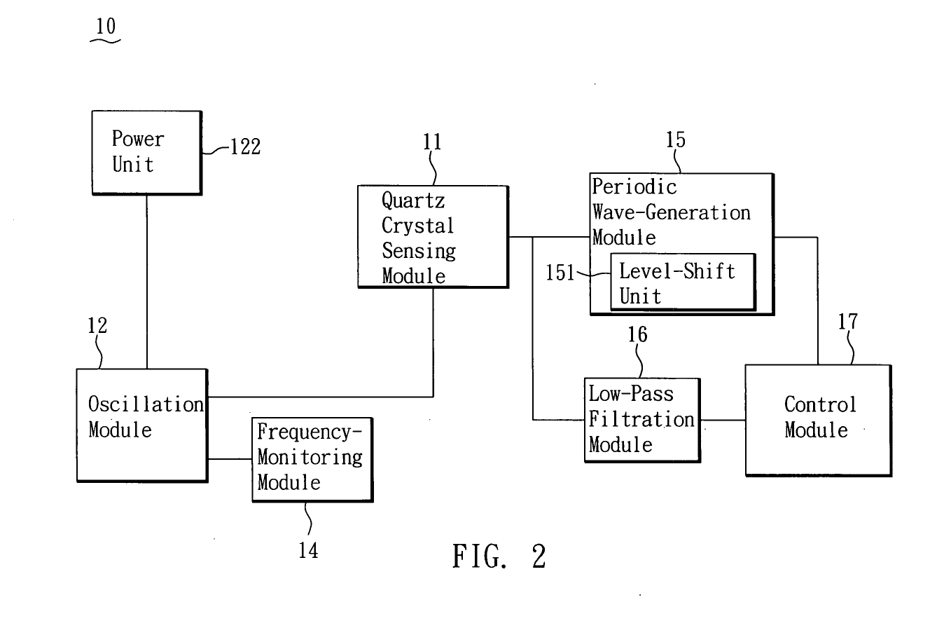

FIG. 2 shows a block diagram of a cell measurement 10 in the present example. The cell measurement 10 includes: a power unit 122, an oscillation module 12, a frequency-monitoring module 14, a quartz crystal sensing module 11, a periodic wave-generation module 15, a low-pass filtration module 16, and a control module 17.

The cell measurement system of the present invention is substantially similar to that of Example 1 except for the following member. A level-shift unit 151 is integrated in the periodic wave-generation module 15. When the control module 17 outputs a starting signal to the periodic wave-generation module 15, the first periodic wave of which the middle voltage level is shifted down can be directly transmitted to a cell sample to be tested because the periodic wave-generation module 15 has the level-shift unit 151.

### Example 3

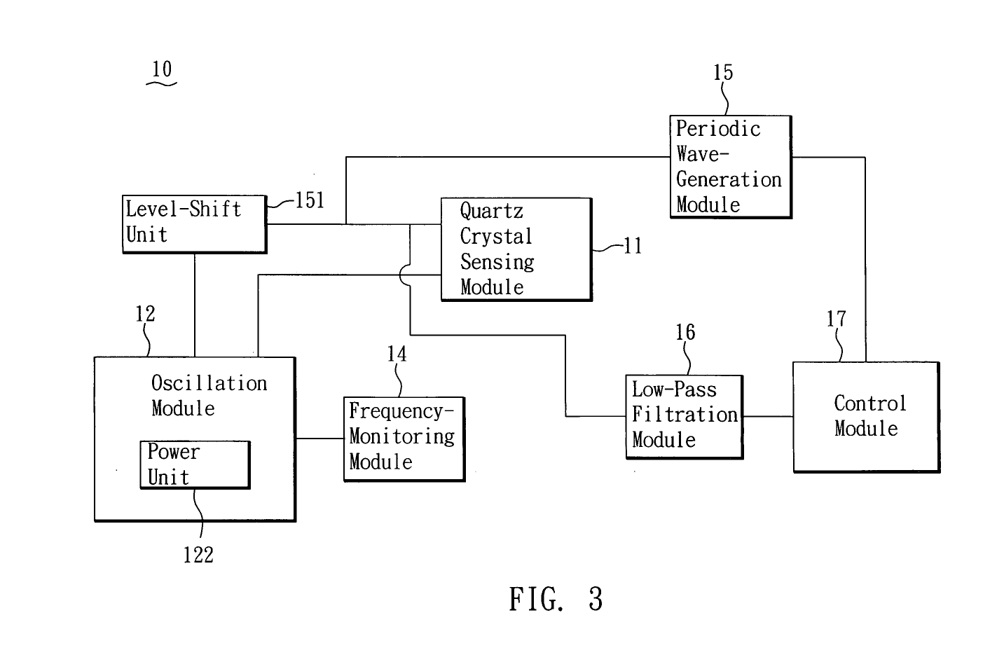

FIG. 3 shows a block diagram of a cell measurement 10 in the present example. The cell measurement 10 includes: an oscillation module 12, a frequency-monitoring module 14, a level-shift unit 151, a quartz crystal sensing module 11, a periodic wave-generation module 15, a low-pass filtration module 16, and a control module 17.

The cell measurement system of the present invention is substantially similar to that of Example 1 except for the following member. A power unit 122 is integrated in the oscillation module 12. Therefore, the oscillation module 12 having the power unit 122 can directly make the quartz crystal of the quartz crystal sensing module 11 resonate.

### Example 4

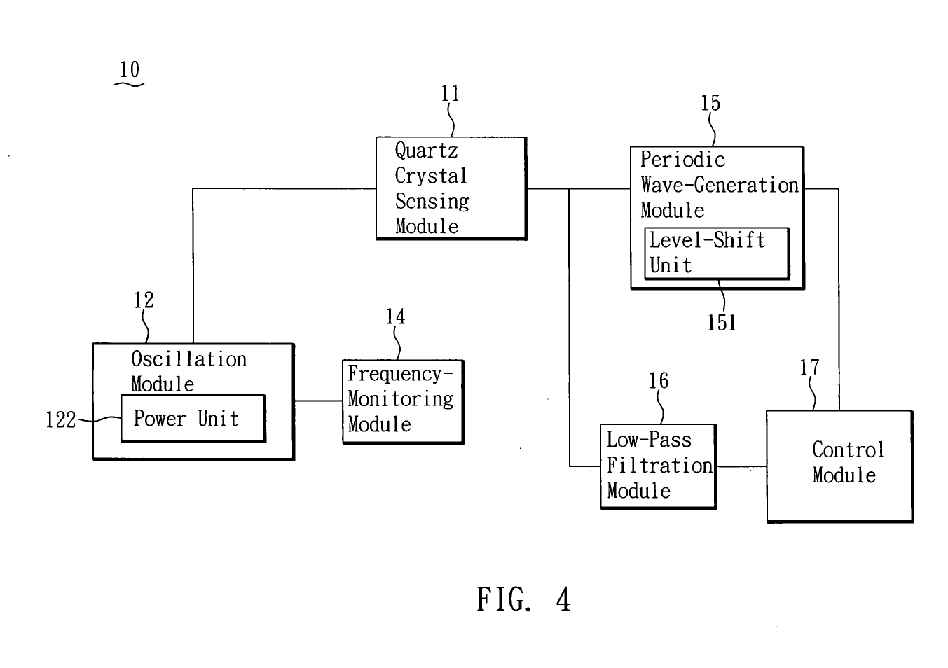

FIG. 4 shows a block diagram of a cell measurement 10 in the present example. The cell measurement 10 includes: an oscillation module 12, a frequency-monitoring module 14, a quartz crystal sensing module 11, a periodic wave-generation module 15, a low-pass filtration module 16, and a control module 17.

The cell measurement system of the present invention is substantially similar to that of Example 1 except for the following members. A power unit 122 is integrated in the oscillation module 12, and a level-shift unit 151 is integrated in the periodic wave-generation module 15. Therefore, the oscillation module 12 having the power unit 122 can directly make the quartz crystal of the quartz crystal sensing module 11 resonate, and the periodic wave-generation module 15 having the level-shift unit 151 can transmit the first periodic wave of which the middle voltage level is shifted down to a cell sample to be tested.

### Example 5

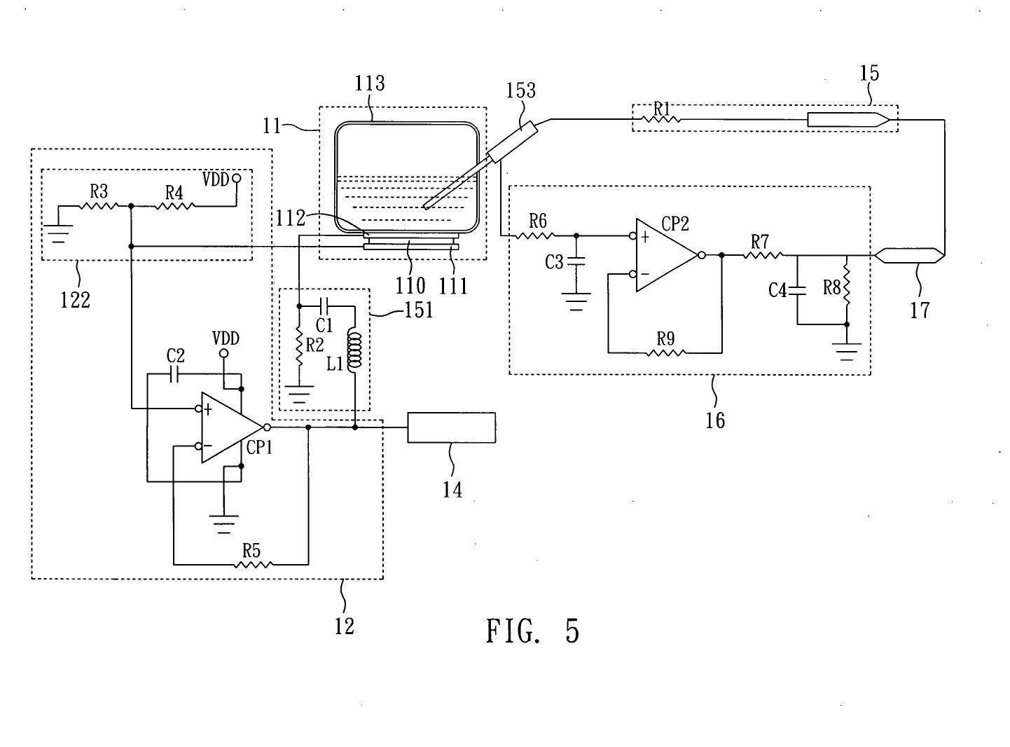

FIG. 5 shows a circuit diagram of the cell measurement system of Example 3. As shown in FIG. 5, the oscillation module 12 includes the power unit 122, the level-shift unit 151, a comparator CP1, a capacitor C2, a device power VDD, and a resistor R5. The frequency-monitoring module 14 is coupled to an output end of the comparator CP1 in the oscillation module 12.

The power unit 122 includes a resistor R3, a resistor R4, and another device power VDD. One end of the resistor R4 is connected to the device power VDD and the other end thereof is connected to one end of the resistor R3. The other end of the resistor R3 is connected to a low potential.

The level-shift unit 151 includes a resistor R2, a capacitor C1, and an inductor L1. One end of the capacitor C1 is connected to one end of the resistor R2, and the other end of the capacitor C1 is connected to one end of the inductor L1. The other end of the resistor R2 is connected to a low potential.

The resistor R3 and the resistor R4 of the power unit 122 are connected to a positive input end (+) of the comparator CP1. One end of the capacitor C2 is connected to a pin of the comparator CP1, and the other end thereof is connected to another pin of the comparator CP1. One end of the resistor R5 is connected to a negative input end (+) of the comparator CP1, and the other end thereof is connected to an output end of the comparator CP1 and the other end of the inductor L1 of the level-shift unit 151.

The quartz crystal sensing module 11 includes a first electrode 111, a second electrode 112, a quartz crystal 110 disposed between the first electrode 111 and the second electrode 112, and a sample well 113. The sample well 113 has an opening for the second electrode 112 to contact with a cell sample to be tested and to detect the change of the tested cell sample during cell growth. The first electrode 111 is coupled to the capacitor C1 and the resistor R2 of the level-shift unit 151, and the second electrode 112 is coupled to the resistor R3 and the resistor R4 of the power unit 122.

The control module 17 is coupled to the periodic wave-generation module 15 and the low-pass filtration module 16. The periodic wave-generation module 15 includes a resistor R1 and a third electrode 153, and the third electrode 153 contacts with a cell sample to be tested.

The low-pass filtration module 16 includes a resistor R6, a resistor R7, a resistor R8, a resistor R9, a capacitor C3, a capacitor C4, and a comparator CP2. One end of the capacitor R6 is coupled to the second electrode 112 of the quartz crystal sensing module 11, and the other end thereof is connected to one end of the capacitor C3 and a positive input end (+) of the comparator CP2. The other end of the capacitor C3 is connected to a low potential. One end of the resistor R9 is connected to a negative input end (−) of the comparator CP2, and the other end thereof is connected to an output end of the comparator CP2 and one end of the resistor R7. The other end of the resistor R7 is connected to one end of the resistor R8 and one end of the capacitor. The other end of the resistor R8 is connected to the other end of the capacitor C4 and both are connected to a low potential.

When the control module 17 outputs a starting signal to the periodic wave-generation module 15, the periodic wave-generation module 15 provides a first periodic wave to a cell sample to be tested. The first periodic wave passing through the tested sample is transmitted to the second electrode 112 of the quartz crystal sensing module 11. The signal passing through the low-pass filtration module 16 is formed a second periodic wave. The control module 17 receives and processes the second periodic wave output from the low-pass filtration module 16 to calculate the changes of the frequency and the TEER owing to the tested cell sample.

Accordingly, the frequency of the quartz crystal can be retrieved by a cooperation of the second electrode 112, the first electrode 111 of the quartz crystal sensing module 11, and the oscillation module 12. The TEER can be retrieved by a cooperation of the periodic wave-generation module 15 (including the third electrode 153), the second electrode 112 of the quartz crystal sensing module 11, the level-shift unit 151, and the low-pass filtration module 16.

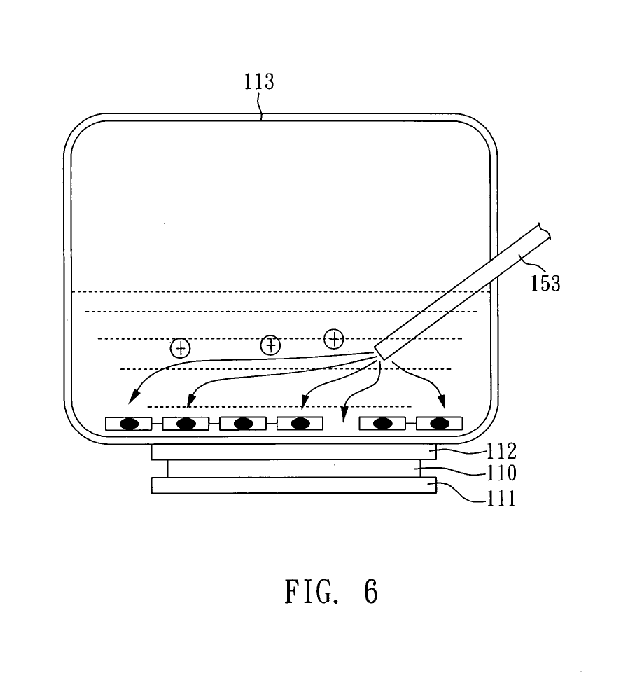

FIG. 6 shows an arrangement of the electrodes for measuring TEER. As shown in FIG. 6, when the third electrode 153 provides the first periodic wave to a cell sample to be tested, the first periodic wave can pass through the tested cell sample because the tested cell sample contains rich ions serving as current channels. However, the cell membranes are constructed of lipid bilayer and functions as a barrier of ionic permeation, and thus the degree of the tight junction between the cells can influence the resistance detected by the second electrode 112. In other words, if there is good tight junction between the cells (i.e. the cell monolayer is complete), it is difficult for the current to pass through and thus the output TEER is relative high. On the contrary, if there is poor tight junction between the cells (i.e. the cell monolayer is incomplete and has openings), it is easy for the current to pass through and thus the output TEER is relative low.

In conclusion, the cell measurement system of the present invention can extend the applications of QCMs and apply QCMs to measure TEER but keep the original performance of QCMs. In addition, consecutive measurement of TEER will not cause deionization or polarization to influence the growth of the tested cells.

Although the present invention has been explained in relation to its preferred embodiment, it is to be understood that many other possible modifications and variations can be made without departing from the scope of the invention as hereinafter claimed.

What is claimed is:

1. A cell measurement system, which measures changes of frequency and transepithelial electrical resistance of a tested cell sample, comprising:
a quartz crystal sensing module having a first electrode, a second electrode, a quartz crystal disposed between the first and second electrodes, and a sample tank, wherein the sample tank is used to receive the tested cell sample to be measured by the second electrode;
an oscillation module coupled to the first and second electrodes of the quartz crystal sensing module to oscillate the quartz crystal thereof;
a periodic wave-generation module coupled to the quartz crystal sensing module and having a third electrode to provide a first periodic wave, wherein the first periodic wave is transmitted to the tested cell sample by the third electrode of the periodic wave-generation module;
a low-pass filtration module coupled to the periodic wave-generation module to receive the first periodic wave transmitted to the tested cell sample and outputting a second periodic wave; and
a control module coupled with the periodic wave-generation module and the low-pass filtration module to allow the periodic wave-generation module to produce the first periodic wave and receiving and processing the second periodic wave output from the low-pass filtration module to calculate the changes of the frequency and the transepithelial electrical resistance of the tested cell sample.

2. The cell measurement system as claimed in claim 1, further comprising: a power unit coupled to the oscillation module and the quartz crystal sensing module to energize the oscillation module and the quartz crystal sensing module.

3. The cell measurement system as claimed in claim 2, further comprising: a level-shift unit coupled to the oscillation module, the quartz crystal sensing module, and the periodic wave-generation module to shift a voltage level of the first periodic wave output by the periodic wave-generation module.

4. The cell measurement system as claimed in claim 1, wherein the oscillation module comprises: a power unit to energize the oscillation module.

5. The cell measurement system as claimed in claim 4, wherein the periodic wave-generation module further comprises: a level-shift unit to shift a voltage level of the first periodic wave output by the periodic wave-generation module.

6. The cell measurement system as claimed in claim 4, further comprising: a level-shift unit coupled to the oscillation module, the quartz crystal sensing module, and the periodic wave-generation module to shift a voltage level of the first periodic wave output by the periodic wave-generation module.

7. The cell measurement system as claimed in claim 1, wherein the periodic wave-generation module comprises: a level-shift unit to shift a voltage level of the first periodic wave output by the periodic wave-generation module.

8. The cell measurement system as claimed in claim 7, further comprising: a power unit coupled to the oscillation module and the quartz crystal sensing module to energize the oscillation module and the quartz crystal sensing module.

9. The cell measurement system as claimed in claim 1, further comprising: a frequency-monitoring module coupled to the oscillation module to monitor a frequency of a voltage level output by the oscillation module.

10. The cell measurement system as claimed in claim 1, wherein the control module comprises: an analog-to-digital converter unit.
